(19) United States
(12) Patent Application Publication
(10) Pub. No.: US 2025/0372215 A1
(43) Pub. Date: Dec. 4, 2025

(54) GENETIC HEALTH INFORMATION SYSTEM

(71) Applicant: INTELLIGENE SA (PTY) LTD, Pretoria (ZA)

(72) Inventors: Heinrich Carl Wilhelm RIX, Pretoria (ZA); McElory Roberto HOFFMANN, Pretoria (ZA); Mathilde Elizabeth VERSTER, Pretoria (ZA)

(73) Assignee: INTELLIGENE SA (PTY) LTD, Pretoria (ZA)

(21) Appl. No.: 18/870,945

(22) PCT Filed: May 31, 2023
(86) PCT No.: PCT/ZA2023/050029

§ 371 Date: Dec. 2, 2024

(30) Foreign Application Priority Data
Jun. 2, 2022 (ZA) 2022/06137

(51) Int. Cl. G16H 10/60; G16H 15/00; G16H 70/40; G16H 70/60; G16H 80/00

(52) U.S. Cl. CPC G16H 10/60; G16H 15/00; G16H 70/40; G16H 70/60; G16H 80/00

(57) ABSTRACT

A genetic health information system includes a repository on which information of a user is stored, the information including genetic information, and an access control means for controlling access to the user's information, the access control means including an authorising means for allowing the user to provide a health practitioner with authorisation to access its information to enable the health practitioner to supply the user with healthcare information based on the genetic information of the user.

### TECHNICAL FIELD

This invention relates to a genetic health information system. In particular, this invention relates to a system for providing genetic and genomic healthcare information.

### SUMMARY OF THE INVENTION

According to the invention, there is provided a genetic health information system including:-

a repository on which information of a user is stored, the information including genetic information; and

an access control means for controlling access to the user's information, the access control means including an authorising means for allowing the user to provide a health practitioner with authorisation to access its information to enable the health practitioner to supply the user with healthcare information based on the genetic information of the user.

The repository may form part of a database of a central processing unit. The database may be in the form of a cloud-based database. The central processing unit may be in the form of a network connected server. The repository may include a user profile or account which may be associated with the user. The information of the user may be stored on the repository against the user profile. The repository may include a health practitioner profile or account which may be associated with the health practitioner. Typically, the repository includes profiles of numerous health practitioners.

The health practitioner may be in the form of any person engaged in human health as a profession. In particular the health practitioner may be in the form of any one or more of the group including, but not limited to, a medical doctor, physician, dietician, optometrist, ophthalmologist, pharmacist, physical therapist, occupational therapist, dentist, psychologist, psychiatrist, sports trainer, genetic specialist, pathologist, nurse practitioner, midwife, chiropractor, speech therapist, audiologist, radiologist, anaesthesiologist, dermatologist, neurologist, cardiologist, pulmonologist, gastroenterologist, endocrinologist, urologist, orthopaedic surgeon, oncologist, rheumatologist, geriatrician, paediatrician, nutritionist, immunologist, allergist, naturopath, homeopath, acupuncturest, reflexologist, and the like.

A profile or account creation means may be provided for allowing profiles of users and/or health practitioners to be created and stored on the repository. The profile creation means may include receiving means for receiving information of the user or health practitioner. The receiving means may be configured to receive any information of the group including user identification information, user genetic information, user medical information, practitioner identification information, practitioner field of practice, practitioner's associated professional governing bodies, and practitioner's professional license number, and the like. It is to be appreciated that the user genetic information may be obtained via a processed DNA sample of the user, the DNA sample typically being in the form of a buccal swab or blood sample, preferably being in the form of a buccal swab. It is to be appreciated further that the processed DNA sample typically produces genetic data capable of being stored on the repository against the user's profile.

The user identification information may include any one or more of the group including identity number, full names, username, medical aid type and number, residential address, user contact details, contact details of third parties such as the user's family or friends, and the like. The contact details may include email addresses and/or phone numbers.

The user medical information may include a historical record of any one or more of the group including medical concerns, illnesses, diagnoses, treatments, prescribed drugs, previous medical practitioners, family medical history, sleep quality, exercise regimes, allergies, vaccination history, surgical procedures, lifestyle habits, dietary information, mental health history, reproductive health, laboratory test results, imaging reports, vital signs, immunizations, alternative medicine usage, genetic test results, social determinants of health, substance use history, environmental exposures, preventive screenings, therapeutic interventions, rehabilitation progress, medication allergies, medical imaging history, organ donation preferences, and the like.

The medical information may include current medical information. The current medical information may include any one or more of the group including current medical concerns, illnesses, diagnoses, treatments, prescribed drugs, exercise regimes, sleep quality, stress levels, blood pressure, blood oxygen levels, heart rate, body temperature, respiratory rate, weight, height, BMI (Body Mass Index), cholesterol levels, blood sugar levels, allergies, immunization status, medication adherence, symptoms, pain levels, mental health status, cognitive function, dietary habits, alcohol or substance use, reproductive health, recent medical procedures, wound healing progress, surgical recovery status, vital signs, laboratory test results, imaging reports, and the like. The current medical information may further include information relating to the nature of the user's employment, typically including a measure of a degree of stress experienced by users in a particular employment type.

The medical information may include activity information. The activity information may include a historical record of overall measurement of activity and/or inactivity, steps, heart rate, exercises, training sessions, specific sports associated with the user, duration of physical activities, calories burned, distance covered, average pace or speed, workout intensity levels, rest periods, sleep patterns, sedentary time, active time, exercise goals and achievements, training plans, fitness assessments, heart rate variability, recovery metrics, exercise equipment usage, workout classes attended, personal bests, performance metrics, exercise-related injuries, exercise frequency, exercise preferences, training load, and the like.

The information of the user may include references to external information which may pertain to the medical information of the user. The references may refer to various health related publications. The health-related publications may be in the form of physical books or textbooks, or in the form of digital publications which may be accessed online. The digital publications may be in the form of medical journals or health-related research, wherein the references may be in the form of links typically accessible by any network connected computing device.

The genetic information included in the user information may be stored on the repository against the user profile. The genetic information may include any one or more of the group including genetic variants, single nucleotide polymorphisms (SNPs), gene expression profiles, chromosomal abnormalities, DNA methylation patterns, epigenetic modifications, copy number variations, mitochondrial DNA data, haplogroups, genetic predispositions, disease-associated genetic markers, carrier status for inherited conditions, pharmacogenomic information, pharmacogenetic information, genotype-phenotype correlations ancestry or ethnic origins, genetic relatedness to other individuals, genetic testing results, genetic counselling records, genotyping or sequencing data, familial genetic history, non-coding RNA analysis, structural variations, gene function annotations, protein-protein interaction networks, pathway analysis results, regulatory elements, disease risk scores, polygenic risk scores, genetic traits or phenotypes, gene ontology annotations, genetic correlations, genome-wide association study (GWAS) results, population-level genetic data, and the like. The pharmacogenetic information may include a comprehensive list of drugs and their effects on the user given the user's specific genotype. The comprehensive list may include information relating to the interaction between a particular drug and the user having a particular genotype. The comprehensive list of drugs may be filtered according to the particular medical information of the user, preferably being filterable according to the current medical information of the user. It is to be appreciated that the current medical information of the user may typically be updated during a consultation with the health practitioner.

An updating means may be provided for allowing the information of the user to be updated. The updating means may be configured to permit manual updating of information. More particularly, manual updating of information may be carried out by an authorised health practitioner. It is to be appreciated that upon consulting with the user, the health practitioner will be required to update the relevant information, including updating of drug prescriptions, symptoms, the presence of illness, and the like. The updating means may be configured to receive results of medical tests for reference thereto by a health practitioner. Further, the updating means may be configured to permit manual updating of user information by an authorised medical aid service provider. The updating means may be configured to permit automatic updating of information. More particularly, information may be automatically updated by obtaining medical or activity information from a smart device capable of measuring health related data, wherein the smart device periodically communicates information updates to the repository.

The access control means may include a login system for allowing access to the information stored on the repository. The login system may be configured to allow the user to access its user profile. The login system may be configured to allow a health practitioner to access any user profile to which access has been duly authorised. The login system may include data input fields for receiving input of an identifier and a verifier. The identifier may be in the form of any of the identification details of the user or health practitioner. The verifier may be in the form of a password or an answer to a security question. The login system may include a secondary verification means which may require input of an OTP (one-time pin) or a TOTP (time-based one-time pin) for in order to permit logging in of the user.

The access control means may include an access means for allowing the user to access the login system and/or the information stored on the user's profile. The access means may be in the form of a dedicated software application which may be stored on the user's personal computing device, preferably being available for download via a suitable app store. Alternatively, the access means may be in the form of a web-based application which may be accessible by an internet browser on any computing device.

The authorising means may be configured to be initiated by a registered user and/or health practitioner. The authorising means may be initiated by a registered health practitioner upon submission of a request to the particular user, typically using an identifier of the user, which request may be sent to and received by the user's linked personal computing device. The request may include a prompt to grant or deny access to the user's information, wherein upon granting of permission, the user is provided with a TOTP to pass on to the health practitioner for input into the system. The request may include identification details of the health practitioner requesting access. Successful entry of the TOTP by the health practitioner typically permits access to the user's profile. The authorisation means may be configured to permit access to the user's profile for a limited time.

Alternatively, the authorising means may be configured to be initiated by the user once logged in. The user may be provided with a list of health practitioners registered to use the health information system. The user may be prompted to select the relevant health practitioner to which the user wishes to grant access. Once selected, the health practitioner may be notified and provided with a link to access the user's information. The notification and link may be sent to the health practitioner's computing device.

The access control means may be configured to permit access to the repository by a system service provider for allowing any system variables or process to be altered or updated as required to ensure proper functioning of the system.

A reporting means may be provided for compiling a report containing any information stored on the user's profile. The report may include a summary of the user's genetic information. The report may include a detailed analysis of the user's genetic information, which detailed analysis may include information relating to influences of a particular user's genome on the user's overall health. The report may include recommended medical treatments or drugs which may be suitable to the particular user. The report may include a comprehensive list of possible drugs to be used along with the corresponding efficacy and potential results in relation to the genetic information of the user. The report may include details of the use of a particular drug by the user having a particular genotype, including precautionary details of the use of the particular drug. The report may include details of alternative drugs which may be used by the user. The report may include a detailed analysis of logic processes utilised to make various recommendations for allowing the health practitioner to view and verify the steps taken to reach a particular recommendation.

The report may be exportable to any suitable file format type for facilitating storage, printing, viewing, and the like. For example, the report may be exportable to PDF-type format to facilitate viewing thereof by the health practitioner or the user, or the report may be exportable to a CSV-type file format for facilitating manipulation thereof by a program capable of carrying out numerical manipulations, such as Microsoft Excel, for example.

A notification means may be provided for notifying the user or the authorised health practitioner when research reveals new genetic information, specifically pharmacogenomic and/or pharmacogenetic information, which would have an influence on the recommendations provided to the user.

A search function may be provided for allowing the authorised health practitioner to search for drugs by name, illness or class, genotype, and treatments which may relate to a health condition of the user. A filtering means may be provided for filtering the results of the search according to a particular gene or any other variable which may relate to the health of the user, such as age for example.

### BRIEF DESCRIPTION OF THE DRAWINGS

A genetic health information system in accordance with the invention will now be described by way of the following, non-limiting examples with reference to the accompanying drawings.

In the drawings:-

FIG. 1 is a flow diagram providing a general overview of the genetic health information system.

### DETAILED DESCRIPTION OF THE INVENTION

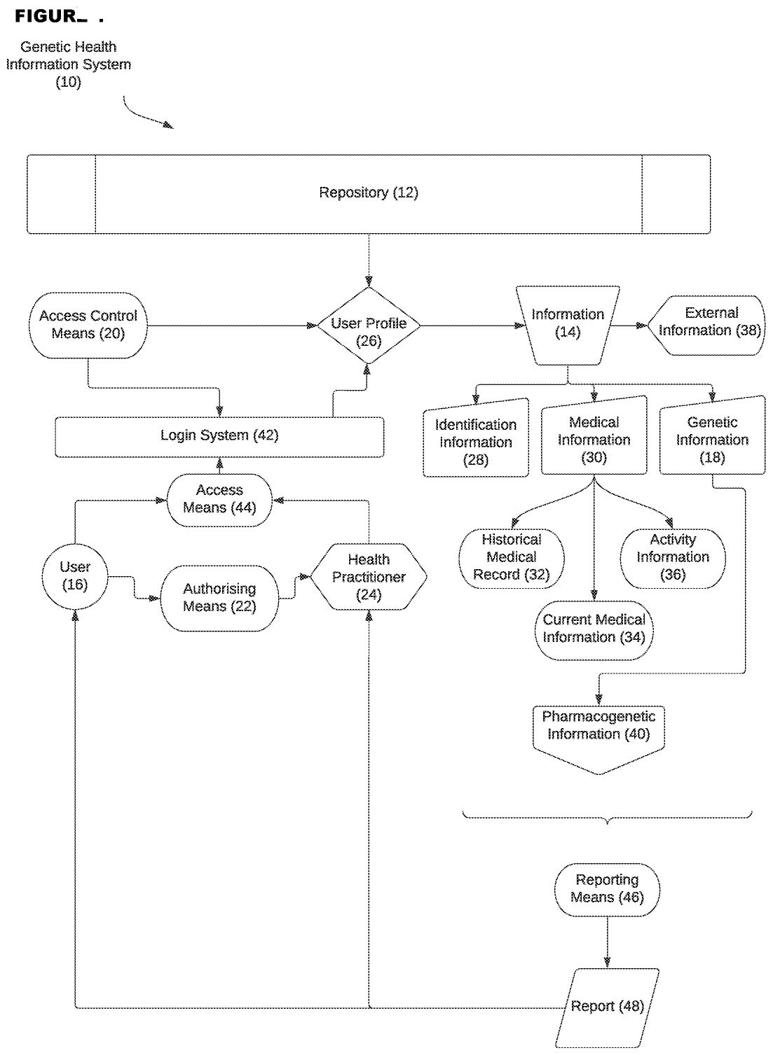

Referring now to FIGS. 1, reference numeral 10 refers generally to a genetic health system in accordance with the present invention. The genetic health information system 10 includes a repository 12 on which information 14 of a user 16 is stored, the information 14 including genetic information 18, and an access control means 20 for controlling access to the user's information 14, the access control means 20 including an authorising means 22 for allowing the user 16 to provide a health practitioner 24 with authorisation to access its information to enable the health practitioner 24 to supply the user 16 with healthcare information based on the genetic information 18 of the user 16.

The repository 12 forms part of a database (not shown) of a central processing unit (not shown). The database (not shown) is in the form of a cloud-based database (not shown). The central processing unit (not shown) is in the form of a network connected server (not shown). The repository 12 includes a user profile or account 26 which is associated with the user 16. The information 14 of the user 16 is typically stored on the repository 12 against the user profile 26. The repository 12 includes a health practitioner profile (not shown) which is associated with the health practitioner 24. Typically, the repository 12 includes profiles of numerous health practitioners.

The health practitioner 24 is in the form of any person engaged in human health as a profession. In particular the health practitioner 24 is typically in the form of any one or more of the group including, but not limited to, a medical doctor, physician, dietician, optometrist, ophthalmologist, pharmacist, physical therapist, occupational therapist, dentist, psychologist, psychiatrist, sports trainer, genetic specialist, pathologist, nurse practitioner, midwife, chiropractor, speech therapist, audiologist, radiologist, anaesthesiologist, dermatologist, neurologist, cardiologist, pulmonologist, gastroenterologist, endocrinologist, urologist, orthopaedic surgeon, oncologist, rheumatologist, geriatrician, paediatrician, nutritionist, immunologist, allergist, naturopath, homeopath, acupuncturest, reflexologist, and the like.

A profile or account creation means (not shown) is provided for allowing profiles to be created, typically user profiles 26 and health practitioner profiles (not shown), and stored on the repository 12. The profile creation means (not shown) includes a receiving means (not shown) for receiving information of the user or health practitioner. The receiving means is configured to receive any information of the group including user identification information 28, user genetic information 18, user medical information 30, practitioner identification information, practitioner field of practice, practitioner's associated professional governing bodies, and practitioner's professional license number, and the like. It is to be appreciated that the user genetic information 18 is obtained via a processed DNA sample of the user 16, the DNA sample typically being in the form of a buccal swab or blood sample, typically being in the form of a buccal swab. It is to be appreciated further that the processed DNA sample typically produces genetic data capable of being stored on the repository 12 against the user's profile 26.

The user identification information 28 includes any one or more of the group including identity number, full names, username, medical aid type and number, residential address, user contact details, contact details of third parties such as the user's family or friends, and the like. The contact details includes email addresses and phone numbers.

The user medical information 30 includes a historical record 32 of any one or more of the group including medical concerns, illnesses, diagnoses, treatments, prescribed drugs, previous medical practitioners, family medical history, sleep quality, exercise regimes, allergies, vaccination history, surgical procedures, lifestyle habits, dietary information, mental health history, reproductive health, laboratory test results, imaging reports, vital signs, immunizations, alternative medicine usage, genetic test results, social determinants of health, substance use history, environmental exposures, preventive screenings, therapeutic interventions, rehabilitation progress, medication allergies, medical imaging history, organ donation preferences, and the like.

The user medical information 30 includes current medical information 34. The current medical information 34 includes any one or more of the group including current medical concerns, illnesses, diagnoses, treatments, prescribed drugs, exercise regimes, sleep quality, stress levels, blood pressure, blood oxygen levels, heart rate, body temperature, respiratory rate, weight, height, BMI (Body Mass Index), cholesterol levels, blood sugar levels, allergies, immunization status, medication adherence, symptoms, pain levels, mental health status, cognitive function, dietary habits, alcohol or substance use, reproductive health, recent medical procedures, wound healing progress, surgical recovery status, vital signs, laboratory test results, imaging reports, and the like. The current medical information 34 further includes information relating to the nature of the user's employment, typically including a measure of a degree of stress experienced by users in a particular employment type.

The user medical information 30 includes activity information 36. The activity information 36 includes a historical record of overall measurement of activity or inactivity, steps, heart rate, exercises, training sessions, specific sports associated with the user, duration of physical activities, calories burned, distance covered, average pace or speed, workout intensity levels, rest periods, sleep patterns, sedentary time, active time, exercise goals and achievements, training plans, fitness assessments, heart rate variability, recovery metrics, exercise equipment usage, workout classes attended, personal bests, performance metrics, exercise-related injuries, exercise frequency, exercise preferences, training load, and the like.

The information 14 relating to the user 16 includes references to external information 38 which pertains to the medical information 30 of the user. The references typically refer to various health related publications (not shown). The health-related publications (not shown) are in the form of physical books or textbooks, or in the form of digital publications which is accessed online. The digital publications are typically in the form of medical journals or health-related research, wherein the references are in the form of links typically accessible by any network connected computing device (not shown).

The genetic information 18 included in the user information 14 is stored on the repository 12 against the user profile 26. The genetic information 18 includes any one or more of the group including genetic variants, single nucleotide polymorphisms (SNPs), gene expression profiles, chromosomal abnormalities, DNA methylation patterns, epigenetic modifications, copy number variations, mitochondrial DNA data, haplogroups, genetic predispositions, disease-associated genetic markers, carrier status for inherited conditions, pharmacogenomic information, pharmacogenetic information 40, genotype-phenotype correlations ancestry or ethnic origins, genetic relatedness to other individuals, genetic testing results, genetic counselling records, genotyping or sequencing data, familial genetic history, non-coding RNA analysis, structural variations, gene function annotations, protein-protein interaction networks, pathway analysis results, regulatory elements, disease risk scores, polygenic risk scores, genetic traits or phenotypes, gene ontology annotations, genetic correlations, genome-wide association study (GWAS) results, population-level genetic data, and the like. The pharmacogenetic information 40 includes a comprehensive list of drugs and their effects on the user given the user's specific genotype. The comprehensive list includes information relating to the interaction between a particular drug and the user 16 having a particular genotype. The comprehensive list of drugs can be filtered according to the particular medical information 30 of the user 16, typically being filterable according to the current medical information 34 of the user 16. It is to be appreciated that the current medical information 34 of the user 16 can be updated during a consultation with an authorised health practitioner 24.

An updating means (not shown) is provided for allowing the information 14 relating to the user 16 to be updated. The updating means (not shown) is configured to permit manual updating of information. More particularly, manual updating of information is carried out by an authorised health practitioner 24. It is to be appreciated that upon consulting with the user 16, the health practitioner 24 will be required to update the relevant information, including updating of drug prescriptions, symptoms, the presence of illness, and the like. The updating means (not shown) is configured to receive results of medical tests for reference thereto by the health practitioner 24 or a subsequent health practitioner. Further, the updating means (not shown) is configured to permit manual updating of user information 14 by an authorised medical aid service provider (not shown). The updating means (not shown) is configured to permit automatic updating of information. More particularly, automatic updating of information is achieved by obtaining medical or activity information from a smart device (not shown) capable of measuring health related data, wherein the smart device (not shown) periodically communicates information updates to the repository 12.

The access control means 20 includes a login system 42 for allowing access to the information stored on the repository 12. The login system 42 is configured to allow a registered user 16 to access its user profile 26. The login system is configured to allow a registered health practitioner 24 to access any user profile 26 to which access has been duly authorised. The login system 42 includes data input fields (not shown) for receiving input of an identifier (not shown) and a verifier (not shown). The identifier (not shown) is typically in the form of any of the identification details of the user 16 or health practitioner 24. The verifier (not shown) is in the form of a password or an answer to a security question. The login system 42 includes a secondary verification means (not shown) which may require input of an OTP (one-time pin) or a TOTP (time-based one-time pin) for in order to permit logging in of the user 16 or the health practitioner 24.

The access control means 20 includes an access means 44 for allowing the user or authorised health practitioner 24 to access the login system 42 and the information 14 stored on the user's profile 26. The access means 44 is in the form of a dedicated software application (not shown) which is stored on the user's personal computing device (not shown). Alternatively, the access means 44 is in the form of a web-based application (not shown) which is accessible by an internet browser (not shown) on any computing device (not shown).

The authorising means 22 is configured to be initiated by a registered health practitioner 24. The registered health practitioner 24 typically submits a request (not shown) to the particular user 16, typically using an identifier, such as an identity number, of the user 16, which request is sent to and received by the user's linked personal computing device (not shown). The request (not shown) includes a prompt (not shown) to grant or deny access to the user's information, wherein upon granting of permission, the user 16 is provided with a TOTP to pass on to the health practitioner 24 for input into the system. The request (not shown) typically includes identification details (not shown) of the health practitioner 24 requesting access. Upon successful entry of the TOTP by the health practitioner 24, access to the user's profile 26 is permitted. The authorisation means 22 can be configured to permit access to the user's profile 26 for a limited time.

The access control means 20 is configured to permit access to the repository 12 by a system service provider (not shown) for allowing any system variables (not shown) or process (not shown) to be altered or updated as required to ensure proper functioning of the system 10.

A reporting means 46 is provided for compiling a report 48 containing any information 14 stored on the user's profile 26. The report 48 can be compiled to display any information 14 stored against the user's profile 26. The below are examples of what the report 48 is capable of displaying, but it is appreciated that the report contents may be altered as desired. The report 48 can include a summary of the user's genetic information 18. The report 48 can include a detailed analysis of the user's genetic information 18, which detailed analysis includes information relating to influences of a particular user's genome on the user's overall health. The report 48 can include recommended medical treatments or drugs which are suitable to the particular user 16. The report 48 can include a comprehensive list of possible drugs to be used along with the corresponding efficacy and potential results in relation to the genetic information 18 of the user. The report 48 can include details of the use of a particular drug by the user 16 having a particular genotype, including precautionary details of the use of the particular drug. The report 48 can include details of alternative drugs which can be used by the user 16. The report 48 can include a detailed analysis of logic processes utilised to make various recommendations for allowing the health practitioner 24 to view and verify the steps taken to reach a particular recommendation.

The report 48 is exportable to any suitable file format type for facilitating storage, printing, viewing, and the like. For example, the report is exportable to PDF-type format to facilitate viewing thereof by the health practitioner or the user, or the report is exportable to a CSV-type file format for facilitating manipulation thereof by a program capable of carrying out numerical manipulations, such as Microsoft Excel, for example.

A notification means (not shown) is provided for notifying the user 16 or an authorised health practitioner 24 when research reveals new genetic information 18, typically pharmacogenomic or pharmacogenetic information 40, which would have an influence on the recommendations provided to the user 16.

A search function (not shown) is provided for allowing an authorised health practitioner 24 to search for drugs by name, illness or class, genotype and treatments which may relate to a health condition of the user 16. A filtering means (not shown) is provided for filtering the results of the search according to a particular gene or any other variable which may relate to the health of the user 16, such as age for example.

It is, of course, to be appreciated that the genetic health information system in accordance with the invention is not limited to the precise constructional and functional details as hereinbefore described with reference to the accompanying drawings and which may be varied as desired.

Although only certain embodiments of the invention have been described herein, it will be understood by any person skilled in the art that other modifications, variations, and possibilities of the invention are possible. Such modifications, variations and possibilities are therefore to be considered as falling within the spirit and scope of the invention and hence form part of the invention as herein described and/or exemplified. It is further to be understood that the examples are provided for illustrating the invention further and to assist a person skilled in the art with understanding the invention and is not meant to be construed as unduly limiting the reasonable scope of the invention.

The inventor believes that the genetic health system in accordance with the present invention is advantageous in that users are capable of being made aware of and accessing personal and valuable genetic related health information. Access to such information is believed to improve the accuracy and efficacy of medical advice. Further, the invention is believed to reduce a likelihood of negative consequences as a result of providing the user with a genetically unsuitable drug.

1-25. (canceled)

26. A genetic health information system including:-
a repository on which information of a user is stored, the information including genetic information;
an access control means for controlling access to the user's information, the access control means including an authorising means for allowing the user to provide a health practitioner with authorisation to access its information to enable the health practitioner to supply the user with healthcare information based on the genetic information of the user; and
a profile creation means for allowing profiles for users to be created and stored on the repository, wherein the profile creation means includes receiving means for receiving any information of the group including user identification information, user genetic information, user medical information, practitioner identification information, practitioner field of practice, practitioner's associated professional governing bodies, and practitioner's professional license number.

27. A genetic health information system as claimed in claim 26 wherein the profile creation means is configured to allow profiles for health practitioners to be created and stored on the repository.

28. A genetic health information system as claimed in claim 26, wherein the user medical information includes historical medical information of any one or more of the group including medical concerns, illnesses, diagnoses, treatments, prescribed drugs, previous medical practitioners, family medical history, sleep quality, exercise regimes, allergies, vaccination history, surgical procedures, lifestyle habits, dietary information, mental health history, reproductive health, laboratory test results, imaging reports, vital signs, immunizations, alternative medicine usage, genetic test results, social determinants of health, substance use history, environmental exposures, preventive screenings, therapeutic interventions, rehabilitation progress, medication allergies, medical imaging history, and organ donation preferences.

29. A genetic health information system as claimed in claim 26 wherein the user medical information includes current medical information of any one or more of the group including current medical concerns, illnesses, diagnoses, treatments, prescribed drugs, exercise regimes, sleep quality, stress levels, blood pressure, blood oxygen levels, heart rate, body temperature, respiratory rate, weight, height, BMI (Body Mass Index), cholesterol levels, blood sugar levels, allergies, immunization status, medication adherence, symptoms, pain levels, mental health status, cognitive function, dietary habits, alcohol or substance use, reproductive health, recent medical procedures, wound healing progress, surgical recovery status, vital signs, laboratory test results, and imaging reports.

30. A genetic health information system as claimed in claim 26, wherein the user medical information includes activity information which includes a historical record of overall measurement of activity or inactivity, steps, heart rate, exercises, training sessions, specific sports associated with the user. duration of physical activities, calories burned, distance covered, average pace or speed, workout intensity levels, rest periods, sleep patterns, sedentary time, active time, exercise goals and achievements, training plans, fitness assessments, heart rate variability, recovery metrics, exercise equipment usage, workout classes attended, personal bests, performance metrics, exercise-related injuries, exercise frequency, exercise preferences, and training load.

31. A genetic health information system as claimed in claim 26, wherein the information of the user includes references to external information which pertain to the medical information of the user.

32. A genetic health information system as claimed in claim 26, wherein the genetic information includes any one or more of the group including genetic variants, single nucleotide polymorphisms (SNPs), gene expression profiles, chromosomal abnormalities, DNA methylation patterns, epigenetic modifications, copy number variations, mitochondrial DNA data, haplogroups, genetic predispositions, disease-associated genetic markers, carrier status for inherited conditions, pharmacogenomic information, pharmacogenetic information, genotype-phenotype correlations ancestry or ethnic origins, genetic relatedness to other individuals, genetic testing results, genetic counselling records, genotyping or sequencing data, familial genetic history, non-coding RNA analysis, structural variations, gene function annotations, protein-protein interaction networks, pathway analysis results, regulatory elements, disease risk scores, polygenic risk scores, genetic traits or phenotypes, gene ontology annotations, genetic correlations, genome-wide association study (GWAS) results, and population-level genetic data.

33. A genetic health information system as claimed in claim 32, wherein the pharmacogenetic information includes a comprehensive list of drugs and their effects on the user given the user's specific genotype.

34. A genetic health information system as claimed in claim 26, wherein information of the user is automatically updated by obtaining medical or activity information from a smart device capable of measuring health related data, wherein the smart device periodically communicates information updates to the repository.

35. A genetic health information system as claimed in claim 26, wherein the access control means includes a login system for allowing access to the information stored on the repository, wherein the login system is configured to allow a health practitioner to access any user profile to which access has been duly authorised.

36. A genetic health information system as claimed in claim 26 wherein the authorising means is initiated by a registered health practitioner upon submission of a request to the particular user, using an identifier of the user, which request is sent to and received by the user's linked personal computing device.

37. A genetic health information system as claimed in claim 36 wherein the request includes a prompt to grant or deny access to the user's information, wherein upon granting of permission, the user is provided with a TOTP to pass on to the health practitioner for input into the system.

38. A genetic health information system as claimed in claim 36 wherein the request includes identification details of the health practitioner requesting access.

39. A genetic health information system as claimed in claim 37 wherein successful entry of the TOTP by the health practitioner permits access to the user's profile.

40. A genetic health information system as claimed in claim 26 wherein the authorisation means is configured to permit access to the user's profile for a limited time.

41. A genetic health information system as claimed in claim 26, including a reporting means for compiling a report containing any information stored on the user's profile.

42. A genetic health information system as claimed in claim 41, wherein the report includes any one or more of the group including: a comprehensive list of possible drugs to be used along with the corresponding efficacy and potential results in relation to the genetic information of the user; details of the use of a particular drug by the user having a particular genotype, including precautionary details of the use of the particular drug; details of alternative drugs which are used by the user; and a detailed analysis of logic processes utilised to make various recommendations for allowing the health practitioner to view and verify the steps taken to reach a particular recommendation.

43. A genetic health information system as claimed in claim 26, including a notification means for notifying the user or the authorised health practitioner when research reveals new genetic information which would have an influence on the recommendations provided to the user.

44. A genetic health information system including:-
a repository on which information of a user is stored, the information including genetic information; and
an access control means for controlling access to the user's information, the access control means including an authorising means for allowing the user to provide a health practitioner with authorisation to access its information to enable the health practitioner to supply the user with healthcare information based on the genetic information of the user, wherein information of the user is automatically updated by obtaining medical or activity information from a smart device capable of measuring health related data, wherein the smart device periodically communicates information updates to the repository.

45. A genetic health information system including:-
a repository on which information of a user is stored, the information including genetic information; and
an access control means for controlling access to the user's information, the access control means including an authorising means for allowing the user to provide a health practitioner with authorisation to access its information to enable the health practitioner to supply the user with healthcare information based on the genetic information of the user, wherein the authorising means is initiated by a registered health practitioner upon submission of a request to the particular user, using an identifier of the user, which request is sent to and received by the user's linked personal computing device, wherein the request includes a prompt to grant or deny access to the user's information, wherein upon granting of permission, the user is provided with a TOTP to pass on to the health practitioner for input into the system.
